(19) United States
(12) Patent Application Publication
(10) Pub. No.: US 2004/0228891 A1
(43) Pub. Date: Nov. 18, 2004

(54) Method of and an apparatus for determining a value of a skin characteristic for application of permanent make-up or tattooing to the skin

(76) Inventor: Jorn Kluge, Teltow (DE)

Correspondence Address: SMITH PATENT OFFICE, 1901 PENNSYLVANIA AVENUE N W, SUITE 200, WASHINGTON, DC, 20006

(21) Appl. No.: 10/786,469

(22) Filed: Feb. 26, 2004

(30) Foreign Application Priority Data
Feb. 26, 2003 (DE) 103 08 480.0
Feb. 10, 2004 (DE) 10 2004 006 500.4

(51) Int. Cl. A61K 7/00

(52) U.S. Cl. 424/401

(57) ABSTRACT

The invention relates to a method of and an apparatus for determining a value of a skin characteristic, especially of a type of skin, for the application of permanent make-up or tattooing to the skin. The method comprises the following steps: generating light rays (2) by means of a light source (1); irradiating a test sector (3) of a skin (4), to which tattooing or permanent make-up is to be applied by means of color, with the light rays (2); measuring test light rays (5) formed in the test sector (3) by irradiation with the light rays (2), by a detector means (6) to generate measured electrical test light values of the test light rays (5); processing the measured electrical values by an electronic processing means (7) to determine a characteristic value which is a measure of a characteristic of the skin (4) in the test sector (3) and which is to be taken into account when applying permanent make-up or tattooing to the skin (4); and outputting the characteristic value via an output means (8).

The present invention relates to the determination of characteristics of skin based on measured values, for example, determining the skin type of living creatures, especially human beings, in connection with the application of tattooing or permanent make-up to the skin.

A method with which remission measurements are used to determine the chrominance carrier density in skin is known from publication DE 38 27 457 A1. With this method, at least two constituents of the optical spectrum are measured and the resulting values evaluated by way of a test value processing means.

It is desirable to find out characteristics of the skin for cosmetic applications, such as the application of tattooing or permanent make-up, as those characteristics permit conclusions to be drawn regarding the colored appearance of the skin. The hue of human skin essentially depends on the blood flow in the skin, the amount of melanin, and the thickness of the various dermal layers. Pigments embedded in the epidermis absorb light and UV-radiation.

In view of the fact that the skin of different people has different, individual characteristics, an application of tattoos or permanent make-up is faced with the problem of selecting the color suitable for the desired appearance of the tattoo or permanent make-up. The choice of suitable color is determined to a great deal by the hue of the skin to which the tattooing or permanent make-up is to be applied. Furthermore, however, the appearance of the tattoo or permanent make-up, once applied, is determined by other skin characteristics, too, such as the thickness of the various integument layers, the pH, and the like. The pigmentation of the skin sector of interest is particularly significant because the effect of the hue of the skin that is relevant for the colored appearance of the tattoo or permanent make-up and the effect of the color to be introduced and deposited in the dermis, with the tattooing or application of permanent make-up, become superimposed.

It is, therefore, an object of the instant invention to provide a method of and an apparatus for determining a value of a skin characteristic when applying tattooing or permanent make-up to the skin, both enabling a reliable analysis of the skin in a test sector to be made at low cost and a determination of a characteristic value to be derived from the analysis.

The object is met, in accordance with the invention, by a method as recited in independent claim 1 and an apparatus as recited in independent claim 12.

The invention comprises the concept of irradiating a test sector of a skin to which tattooing or permanent make-up is to be applied, by means of color, with light rays generated by a light source in order thus to determine a value of a skin characteristic for the application of tattooing or permanent make-up. Test light rays in the test sector resulting from the incident light rays are detected by a detector means in order to generate measured electrical test light values. In this manner, optical properties of the test sector of the skin are examined with the aid of light. Test light rays may comprise light components which are influenced by the absorption behavior, the emission behavior, the reflection behavior, and/or the dispersion behavior of the skin in the test sector. The measured test light values thus obtained of the test light rays are processed automatically by an electronic processing means in order to find out a characteristic value which indicates a characteristic of the skin and which must be taken into account when applying tattooing or permanent make-up to the skin. The characteristic value automatically obtained in this manner is output via an output means which, for instance, may be embodied by an electronic display and/or loudspeaker means.

The test light rays preferably are measured in a plurality of optical spectral regions, test light rays being detected in the plurality of spectral regions.

The characteristic value may be output as a skin type value indicating the type of the skin that was examined. Different types of skin are distinguished based on the respective interaction with the incident light rays, especially by absorption and/or reflection. These characteristics of the skin are influenced especially by the individual pigmentation of the skin. A suitable color for the permanent make-up or tattoo may be selected based on the information gathered about the type of skin.

Alternatively or in addition, a chromaticity value is determined as a characteristic value. Preferably, the characteristic value is output in the form of a combination of numbers and/or letters. If the. characteristic value chosen is a chromaticity value it will identify color available from one or more producers of colorants. An output in the form of a chromaticity symbol likewise may be provided. A chromaticity value also may be a chromaticity correction value to indicate corrective color. The chromaticity value identifies a color/corrective color for tattooing or applying permanent make-up, selected automatically in response to the results of measurements made on the test sector under examination. If the chromaticity correction value is determined alternatively or additionally from the measured test light values it indicates corrective color. The addition of a certain quantity of the corrective color to other color, which in this case serves as a kind of basic color, compensates peculiarities/features of the skin under examination, particularly as regards pigmentation. The resulting color used for tattooing or application of permanent make-up, being a mixture of basic color and corrective color, essentially will bring about a colored appearance corresponding to the color image which would result if the basic color were applied to “neutral skin”. The peculiarities/features of the skin are corrected.

According to an embodiment of the invention it may be provided that, when determining the chromaticity correction value, a corrective color volume statement for a volume amount of corrective color (basic color) is determined together with the chromaticity correction value and output via the output means. For example, a number of droplets or ml of corrective color per volume of basic color may be indicated. The user thus is informed of a certain mixing ratio of corrective color and basic color.

The method and apparatus may be used for determining a characteristic value for any desired skin sectors, especially skin sectors comprising an epidermis, and skin sectors, such as lip sectors which dispose of mucous membranes.

A most extensive optical analysis of the test sector of the skin is achieved, with a convenient further development of the invention, by using white light rays as the light rays. A detailed optical analysis of the characteristics of the skin likewise is obtainable when using monochrome light rays of the red, green, and/or blue optical spectral regions.

The accuracy of determining the characteristic value is improved, with a preferred modification of the invention, by processing electronic data including information on further characteristics of the skin in the test sector, especially the pH, when the chromaticity value/correction value is determined by the electronic processing means.

Characterization of the skin characteristic as regards the colored appearance of the skin, making the skin appear “cold” or “warm”, is obtained, with a convenient further development of the invention, in that a measured value characterizing a property by which the skin in the test sector is dyed blue/orange is taken into account when determining the chromaticity value/correction value.

Further automation of the method is achieved, with a preferred embodiment of the invention, in that the chromaticity value/correction value in the form of electronic chromaticity value data is used as input values for an electronic imaging system which automatically generates at least a partial representation of a living test creature for which the test sector of the skin was examined, taking into account the chromaticity value. The value of the skin type, too, may be coupled, alternatively or additionally, into the electronic imaging system. Thereby the liklihood is increased that the colored appearance, to be achieved by applying the color to the skin, indeed will correspond to the appearance as desired.

The advantages of the invention and of possible further developments thereof discussed above manifest themselves particularly when the skin is analyzed automatically for the application of tattooing or permanent make-up to human skin. For such applications, the information obtained regarding the characteristics of the skin may be utilized to optimize the desired result of the colored designing of the skin.

The invention will be described further, by way of example, with reference to the accompanying drawing, in which:

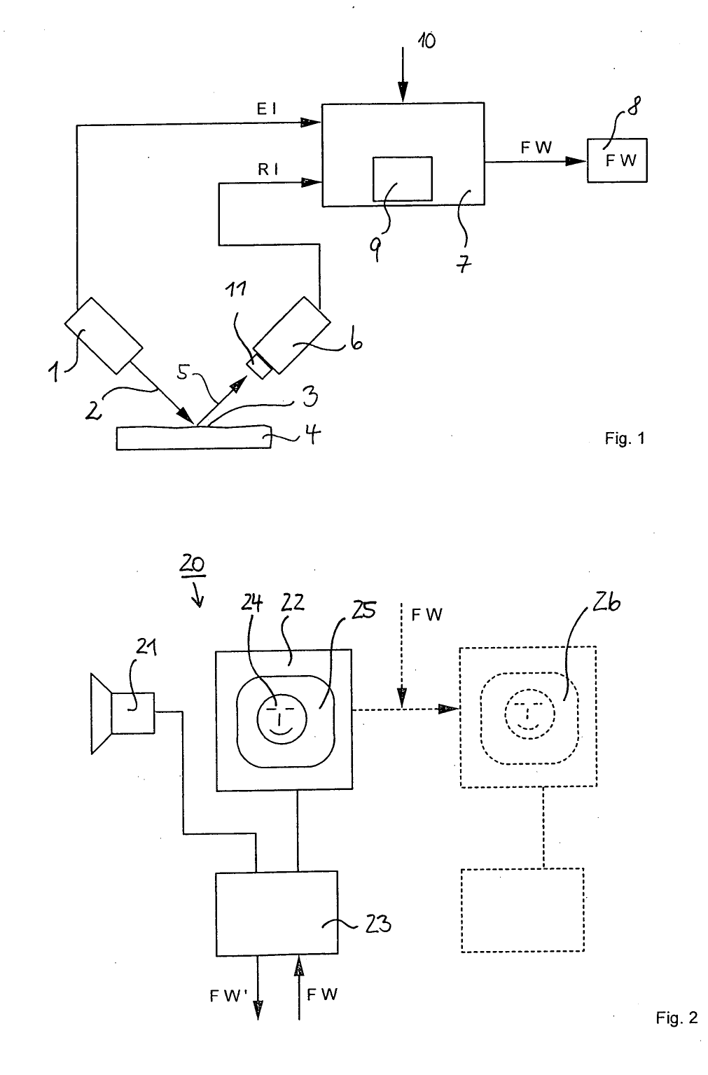

FIG. 1 is a diagrammatic presentation of an apparatus for determining a chromaticity value of color by means of light rays; and

FIG. 2 is a diagrammatic presentation of an arrangement for automatic imaging, automatically taking into account a test value, for example a chromaticity value, in connection with an application of tattooing or permanent make-up.

FIG. 1 diagrammatically shows a light source 1 for generating light rays 2 with which to irradiate a test sector 3 of skin 4. In the test sector 3, the incident light rays 2 at least are partly reflected, absorbed, and/or dispersed, and/or cause emission light whereby test light rays 5 are produced that are detected by a detector means 6. The incident light rays 2 impinge on the test sector 3 at a predetermined irradiation intensity EI. The irradiation intensity EI may be adjusted, for instance, by controlling the voltage supply of the light source 1. Electronic information on the irradiation intensity EI is supplied to an electrical processing means 7. The electronic information on the irradiation intensity EI may be generated, for example, by decoupling part of the incident light rays 2 and directing them to a photodiode (not shown in FIG. 1) to produce an electrical signal which is proportional to the irradiation intensity EI.

The light source 1 used for generating the irradiation light rays 2 basically may be any light source capable of generating monochrome or white light. The spectral half life width of monochrome light rays depends essentially on the light source 1 used. Both narrow-band and broad-band monochrome light rays are useful. A low-cost embodiment makes use of light emitting diodes which generate monochrome light rays in various optical spectral regions. A compact structure is enhanced, with a convenient modification, in that the light source 1 used is a tricolor LED capable of generating monochrome light rays in three different color ranges. This embodiment, moreover, favors cost-saving manufacture of the apparatus.

In the case of white light, a dispersion means 11 is connected upstream of the detector means 6 to cause spectral dispersion of the test light rays 5 so that test values can be determined in various regions of the optical spectrum.

An intensity of the test light rays RI, preferably test light rays in different spectral regions, depends essentially on the extent to which the incident light rays 2 are absorbed, dispersed, and/or reflected by the skin 4 in the test sector 3. A comparison of the intensity of the test light rays RI and the irradiation intensity EI of light rays in different spectral regions, therefore, can provide information on the pigmentation of the skin 4 in the test sector 3. The electronic processing means 7 automatically processes electrical test values of the irradiation intensity EI and the intensity of the test light rays 5 for automatic determination of a measured value of a skin characteristic. In an embodiment for automatic evaluation, an exclusive analysis of the spectral distribution of the test light rays 5 may also be made, disregarding the intensity ratios. Information as to the pigmentation conditions of skin may be gathered from a spectral evaluation. Of course, it is likewise possible to combine spectral evaluation and intensity evaluation.

In principle, any characteristic of the skin influencing the test result of the optical measurement may be examined and processed. The characteristic of the skin 4 under examination thus found out, will be used especially for having the electronic processing means 7 automatically determine a chromaticity value FW and/or a skin type HT for tattooing or application of permanent make-up. To do that, the electronic processing means 7 resorts to electronic data stored either in an internal memory of the electronic processing means 7 or in an external memory connected to the electronic processing means 7. These electronic data permit possible results of measurements gathered in the optical examination of the skin 4 described above to be associated with skin type values/chromaticity values of color so that a chromaticity value FW/skin type value HT can be determined and output automatically in response to the measurement result. As regards the chromaticity value FW, the association allows for the color effect of available color when making use of this color for tattooing/applying permanent make-up to a given type of skin which is characterized by the optical process.

The chromaticity value FW identifies color which is determined in response to the examination of skin described above to achieve a certain color impression in tattooing/applying permanent make-up. The chromaticity value likewise may identify a whole series of colors suitable to produce a respective colored appearance with a particular type of skin that was determined by the optical measuring process, e.g. a chromaticity value of a color/colors that will makethe skin appear “warm” or “cold”.

The chromaticity value FW is output as electronic information by an output means 8 which may be embodied by a display or loudspeaker, for example, and which either may be integrated in the electronic processing means 7 or be provided outside of the same. The electronic processing means 7 used, for example, may be a suitable microprocessor based on conventional circuitry. It would also be possible to devise a PC by suitable programming and interfacing for control of the light source 1 and the detector means 6 and for processing of the electronic test values of the irradiation intensity EI and the intensity of the test light rays RI.

White light or monochrome light rays of the red, green, and blue spectral regions may be used conveniently for irradiation and detection to permit the skin characteristic examined in the test sector 3 to be determined as accurately as possible.

The electronic processing means 7 disposes of a storage means 9. Electronic data of further parameters characterizing the skin 4 may be memorized in the storage means 9. These may be, for instance, the pH, adipose tissue, thickness of the skin 4, and the like. Electronic information of this kind also may be supplied through an interface 10 to the electronic processing means 7 from other equipment which carries out suitable measurements to determine the respective parameter. Furthermore, such information may be detected and processed upon manual input by a user. The electronic data regarding the respective parameters may be taken into consideration automatically by the electronic processing means 7 in determining the chromaticity value FW/skin type value HT.

FIG. 2 is a diagrammatic presentation of an arrangement 20 comprising a photography means 21, for example a digital camera, and a screen means 22, both connected to a control means 23. The arrangement 20 illustrated in FIG. 2 facilitates and accelerates the procedure of the optimized selection of a suitable color for a tattoo or permanent make-up while automatically taking into account characteristics of the skin to be treated. To begin with, the photography means 21 is actuated to take a picture which shows the person 24 wishing to receive a tattoo or permanent make-up, including the skin sector to be treated. The picture first is shown as an original picture 25 on the screen means 22. Subsequently, when generating a modified image 26, electronic information regarding the characteristic value (FW, HT) is taken into account automatically. The screen means 22 is controlled by the control means 23, shown diagrammatically in FIG. 2, to generate the original picture 25 and the modified image 26.

Starting from the modified image 26, a user may process the presentation to incorporate in the image a modified colored appearance of a tattoo or permanent make-up. The chromaticity value FW which also may indicate corrective color, as explained above, is taken into account automatically by considering, with the colors used for the picture, the color identified by the chromaticity value FW for the tattoo/permanent make-up in the modified image 26 when the modified image 26 is output. If the modified image 26 was modified on the basis of inputs by a user the control means 23 automatically determines and outputs a corrected chromaticity value FW′. Thereupon, the color corresponding to the corrected chromaticity value FW′ may be used for applying the tattooing or permanent make-up to the person 24. If the modified image 26 as such pleases the person 24 in terms of colored appearance and the person does not input any corrections the corrected chromaticity value FW′ corresponds to the chromaticity value FW, and the output of the corrected chromaticity value FW′ may be dispensed with. The choice of the colorant matter having been made in the way described makes sure that a colored appearance will result in dependence on the skin type, as selected by the person 24 assisted by the arrangement 20.

The method described with reference to FIG. 2 does not depend on the way in which the previously determined chromaticity value FW or any other skin characteristic (skin type HT) are arrived at. This means that chromaticity values determined according to the method described with reference to FIG. 1 as well as chromaticity values determined by any other ways and means may be used with the instant method. For imaging, the arrangement 20 thus may automatically process not only the chromaticity value, determined in whatever way, but also other parameters which characterize the skin, provided they are available as electronic data to be processed by the control means 23.

The features of the invention disclosed in the specification above, in the claims and drawing may be significant for implementing the invention in its various embodiments, both individually and in any combination.

1-18. (Canceled)

19: A method of determining a value of a skin characteristic, especially of a type of skin, for the application of permanent make-up or tattooing to the skin, the method comprising the following steps:
generating light rays by means of a light source;
irradiating a test sector of a skin, to which tattooing or permanent make-up is to be applied by means of color, with the light rays;
measuring test light rays formed in the test sector by irradiation with the light rays, by a detector means to generate measured electrical test light values of the test light rays;
processing the measured electrical values by an electronic processing means to determine a characteristic value which is a measure of a characteristic of the skin in the test sector and which is to be taken into account when applying permanent make-up or tattooing to the skin; and
outputting the characteristic value via an output means.

20: The method as claimed in claim 19, wherein monochrome light rays of the red, green, and/or blue spectral regions are used as the light rays.

21: The method as claimed in claim 19, wherein white light rays are used as the light rays.

22: The method as claimed in claim 19, wherein a skin type value indicating a type of the skin in the test sector is determined and output as the characteristic value.

23: The method as claimed in claim 19, wherein a color value indicating color suitable for the skin for application of permanent make-up or tattooing to the skin is determined and output as the characteristic value.

24: The method as claimed in claim 23, wherein a chromaticity correction value for a corrective color indicated by the chromaticity correction value is determined as chromaticity value and output via the output means during the automatic processing of the measured test light values by the electronic processing means.

25: The method as claimed in claim 24, wherein a corrective color volume statement for a volume amount of corrective color per volume amount of color is determined together with the chromaticity correction value and output via the output means.

26: The method as claimed in claim 23, wherein electronic data including information on further characteristics of the skin in the test sector, especially a pH, are processed when determining the chromaticity value/chromaticity correction value by the electronic processing means.

27: The method as claimed in claim 23, wherein a test value determined from the measuring light rays and characterizing blue-coloring pigmentation of the skin in the test sector is taken into account when determining the chromaticity value/chromaticity correction value.

28: The method as claimed in claim 23, wherein another test value determined from the measuring light rays and characterizing a property which colors the skin in the test sector orange is taken into account when determining the chromaticity value/chromaticity correction value.

29: The method as claimed in claim 23, wherein the chromaticity value/chromaticity correction value in the form of electronic chromaticity value data is used as input value for an electronic imaging system with which at least a partial representation of a living test creature for which the test sector of the skin was examined is generated automatically on an electronic display, taking into account the chromaticity value/chromaticity correction value.

30: An apparatus for determining a characteristic value of a skin, especially of a type of skin, for the application of permanent make-up or tattooing to the skin, comprising:
a light source for generating light rays;
a detector means for detecting test light rays which are formed by irradiation of a test sector of the skin with the light rays so as to generate respective measured electrical test light values of the test light rays in a plurality of optical spectral regions;
an electronic processing means for automatic processing of the measured test light values to determine a characteristic value which is a measure of a characteristic of the skin in the test sector and which is to be taken into account when applying permanent make-up or tattooing to the skin; and
an output means for outputting the characteristic value.

31: The apparatus as claimed in claim 30, wherein the electronic processing means is configured to determine, as the characteristic value, a skin type value indicating a type of the skin.

32: The apparatus as claimed in claim 30, wherein the electronic processing means is configured to determine, as the characteristic value, a chromaticity value indicating color for application of permanent make-up or tattooing to the skin.

33: The apparatus as claimed in claim 30, wherein the light source comprises a plurality of light emitting diodes for generating monochromatic light rays in the plurality of optical spectral regions.

34: The apparatus as claimed in claim 30, wherein the processing means is coupled to an arrangement for automatic color analysis for application of tattooing or permanent make-up to the skin in order to transfer the characteristic value to the assembly for automatic processing.

35: The apparatus as claimed in claim 34, wherein the arrangement comprises a screen means for presenting electronic image data and a control means for processing the electronic image data in consideration of the characteristic value and for automatically adapting the characteristic value.

36: The apparatus as claimed in claim 30, wherein a dispersion component for spectral dispersion of the measuring light rays is connected upstream of the detector means.
